(19) United States
(12) Patent Application Publication
(10) Pub. No.: US 2020/0327965 A1
(43) Pub. Date: Oct. 15, 2020

(54) SYSTEM AND METHOD OF INTEGRATED PATIENT UNIQUE IDENTITY MANAGEMENT

(71) Applicants: Albert Davydov, Forest Hills, NY (US); Peter Usov, Belle Mead, NJ (US)

(72) Inventors: Albert Davydov, Forest Hills, NY (US); Peter Usov, Belle Mead, NJ (US)

(21) Appl. No.: 16/383,698

(22) Filed: Apr. 15, 2019

(51) Int. Cl. G16H 10/60; G06F 16/25; G06F 16/22

(52) U.S. Cl. CPC G16H 10/60; G06F 16/22; G06F 16/258

(57) ABSTRACT

The present invention is a system and method of integrated person or patient unique identity management based on unique body parts and special patient tag with universal abilities managing patient identity across data sources.

### BACKGROUND OF THE INVENTION

### Field of the Invention

The present invention is in the field of systems and methods to identify personal data. More specifically, the present invention is an integrated personal data unique identity management system and a method to uniquely identify each person within a global computer system.

### Background of the Invention

The present invention is a system and a method of integrated personal data unique identity management based on human biometric data and a special personal tag with universal abilities of managing person's identity across data sources.

Personal data in many industries, and electronic health care data in healthcare, in particular, are always tried to be linked together across multiple systems, including electronic health records (EHRs), patient registries, claims databases. In general, each system assigns its own identifier to each person whose data it maintains. This process makes it difficult to track data across multiple systems and identify duplicate data entry when different systems are linked. Efforts to address this challenge are complicated by the need to protect personal data privacy and security.

Personal Data Global Unique Identification System (PDGUIS) has been defined as the ability to ascertain a distinct, unique identity as expressed by an identifier that is unique within the scope of the exchange network, given characteristics about that identity such as name, date of birth and gender. PDGUIS is the process of accurately and appropriately identifying, tracking, managing, and linking individuals and their digitized health, or other information, often within and across multiple electronic systems.

A related idea is the concept of personal identity integrity defined as the accuracy and completeness of data attached to, or associated with an individual. Efficient identity management leads to high identity integrity. One of the solutions is to assign a personal identifier tag (PIT), to each person's unique body part patterns (some human body part patterns can be subject for unique identification) and share it among data storage facilities. Based on this attribute there are some biometric data collection devices utilized for security identification and authentication. However, each individual device system contains a certain percentage of inaccuracy and has capability issues.

For example fingerprint method, the oldest and the most popular biometric technology evaluation, is only 99.3 percent reliable. Fingerprint databases of U.S. government agencies alone store more than 200 million records. But issues like prints can rub off, or development of callouses over during hard and repetitive labor complicate the read. More effective, in terms of accuracy, are the palm vein biometric devices. But they are also problematic with issues of high pattern template size and high cost. Other methods and devices may seriously limit the possibilities of their applications in global and nationwide identity systems.

From the other side, a simple generation of global unique identification numbers requires a centralized generation system and a distribution system for these numbers. There are also problems of assigning unique numbers to individuals. There are also problems of assigning different numbers to the same individual, or for different individuals assignment of a same number. In such cases, these numbers cannot be used as incidental because they are not synchronized. For example, medical institutions assign erroneously duplicate numbers to one person. Or, in a personal identity fraud, personal identification can be stolen and an identifier (ID) can be assigned to someone else.

Using the above methods simultaneously creates the person's (or in healthcare, patient's) global unique identification system. It combines the biometric data to verify a person's (patient's) identity and assigns the unique individual number which makes the system more accurate and flexible. This approach eliminates most problems described above because the biometrics can prevent individuals from registering in the global unique identification system multiple times and, vice versa, an individual unique number of a person can be retrieved from the global system using biometric data.

Unfortunately, this method also has serious limitations for globalization. The biometric patterns, or templates, must be stored on a central server during enrollment. Direct matching process of biometric patterns, or templates may take a long time. The personal biometric template captured by the biometric device must be sent to the central server that requires good network, internet connectivity and sufficient hardware operating resources.

Accuracy is based on several other factors such as false acceptance rate (FAR), false reject rate (FRR), error rate, identification rate, etc. Significant disadvantage of those limitations is the fact, that the system cannot operate with institutions which do not have internet connection, or internet connection is unstable. In this case, it will be advisable to have a system that can generate a unique person's IDs from personal identification locally without any connection to the central database. Such a system will be more reliable and easier to use in the PDGUIS.

The unique ID can be formed based on individual data of a person such as first, middle and last names, birthday in combination with personal unique body part pattern, or template. On the other hand, personal unique body part pattern, or template can be supplied with an individual person's, or as in healthcare, individual patient tag (IPT), based on individual data of a person.

This approach reduces the personal IDs data fetch duration from the national database and increase accuracy in storing data. However, limitations for the wide application of this system can be lack of accuracy of the processing and measurements issues and a high cost of instruments.

The present invention method and system offers a simple, cost-effective and portable solution with a high accuracy, with a fast search speed for biometric patterns, or templates in the global database storage.

Today, common biometric approaches include facial recognition, fingerprinting and iris scanning. These systems are limited due to their complexity, infringement on privacy, cost, or portability issues.

The present invention uses ear biometrics that has proven to be a unique and viable solution. It does not require particular actions, such as scanning of a part of a body over an authentication device which makes it easier to conduct continuous authentication. The system works everywhere, even when the person is moving. Ears are remarkably consistent unlike faces, they do not change shape with different expressions, or age and remain fixed in the middle of the side of the head against a predictable background.

In the present invention, we have developed an identification algorithm that also shows good scalability of recognition rate with size of a dataset. We then conducted re-recognition and then identification and statistical analyses to identify the accuracy and replicability of our approach. In conclusion, the bend, or flexure of the ear helix was found to be the most reliable anatomical structure that could be served as the base for re-identification.

It is known that individual ear identification rate might vary from 90% to 99.5% for image ray transform methods. In the present invention, our approach is to use both, left and right ears together, and our rate of identification was up to 100%.

The present invention is a simple, cost-effective and portable personal identification method in the global system that allows managing identity across countrywide data sources. It minimizes data management expenses such as storage requirements for the device itself. The personal data will to be stored locally. There is only a simple web camera and a simple image processing application, or a lightweight image processing application necessary.

The present invention can be used in many industries. The examples could be healthcare global ID verification for medical equipment, electronic healthcare (EHR) patient record management and management of patient identity across data sources. The national patient identification system would identify patients, link patient medical records, and allow broad sharing, monitoring, research and analysis of public using computerized medical records linked through the nationwide health information network (NHIN). Other examples may include government organizations, national identity applications, banking and financial institutions and other facilities requiring high security storage. Personal data information exchange enables information sharing across disparate health care applications. It also can be used as a platform for the development of biometric identification application for desktop and mobile devices. System can meet the minimum hardware requirements for current mobile devices on the market today and can be used on many mobile devices such as Android, or the Apple iOS system, or a like.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a system and method of integrated patient, or person unique identity management, in accordance with one embodiment of the present invention.

FIG. 2 illustrates a system and method of integrated patient, or person unique identity management, in accordance with one embodiment of the present invention.

FIG. 3 illustrates a system and method of integrated patient, or person unique identity management, in accordance with one embodiment of the present invention.

Below is a clear example of the invention use in healthcare industry.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted, or simplified in order not to obscure the illustrative embodiments.

Various operations will be described as multiple discrete operations, in turn, in a manner that is most helpful in understanding the present invention; however, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise. The word “patient” is applicable in relation to the healthcare industry, whereas word “personal” is applicable in other industries alike, instead of the word “patient”.

The patient's unique identity is an entity consisting of two independent identification data objects such as patient's ID tag (PIT) formed from person individual data and ears biometric data.

Patient's Identity Tag (PIT)

PIT is a specific integer number which is formed by converting the person's full name, birthday and eye color by special following algorithm:

A person's full name is the set of names by which an individual is known and that can be introduced as a word-group, with the understanding that, taken together, they all relate to that one individual. The full name must be presented from first/given, middle, and last/family/surname.

From the full name string removed all non-alphanumeric characters;

The full name string value converted to uppercase;

The first character's coefficient number is calculated as the sum of the ASCII values of the first letters of each word in the full name string.

From the full name string removed all spaces;

The first part of the PIT number consists of the sum of ASCII value of each character in a full name string;

The second part of patent's PIT number consists a string join of values from the day number plus the month number and the year of the birthday.

The third part of the patent's PIT number is formed from the length value of the full name string plus first character's coefficient number;

The forth part of the patient's PIT number is an eye color code based on eye color chart:

BLK Black=1

BLU Blue=2

BRO Brown=3

GRY Gray=4

GRN Green=5

HAZ Hazel=6

MAR Maroon=7

PNK Pink=8

DIC Dichromatic=9

SPC Spectrum=10

For example, for the person with full name “James Bob Smith”, date of birth May 15, 1926 and eye color in gray, the PIT will be: 9681926515132234, where:

968—first part of the patent's PIT number consists of the sum of ASCII value of each character in a full name string;

1926515—second part of PIT number consists a string join of values from the day number plus the month number and the year of the Birthday.

13223—third part of the PIT number is formed from the length value (=13) of the full name string and joined first character's coefficient number (74(J)+66(B)+83(S)=223).

4—forth part of the patent's PIT is eye color;

Ear images can be acquired in a similar manner to face images, and a number of researchers have suggested that the human ear is unique enough to each individual to allow practical use as a biometric. For obtaining biometric data of a person's ear can be used a simple video or digital camera. Ear biometric system consists of ear detection and ear recognition modules.

There are several algorithms for image digitizing of human ear. Most popular shape-finding algorithm called “image ray transform,” which boasts 99.6 percent accuracy, according to a study presented at the IEEE Fourth International Conference on Biometrics Sep. 29, 2014. The outer ear may prove to be one of the most accurate and least intrusive ways to identify people.

We propose that the analysis of the curve of the ear's helix to be the most reliable anatomical structure (antihelix, tragus, antitragus, inter-tragic incisura, and the ear lobule) when both left and right ears were paired together. In this approach rate of identification should be 100%.

But since the unique identification of a person consists of two independent identification data objects the requirements to high accuracy can be reduced. This approach boosts performance and reduces costs of measuring.

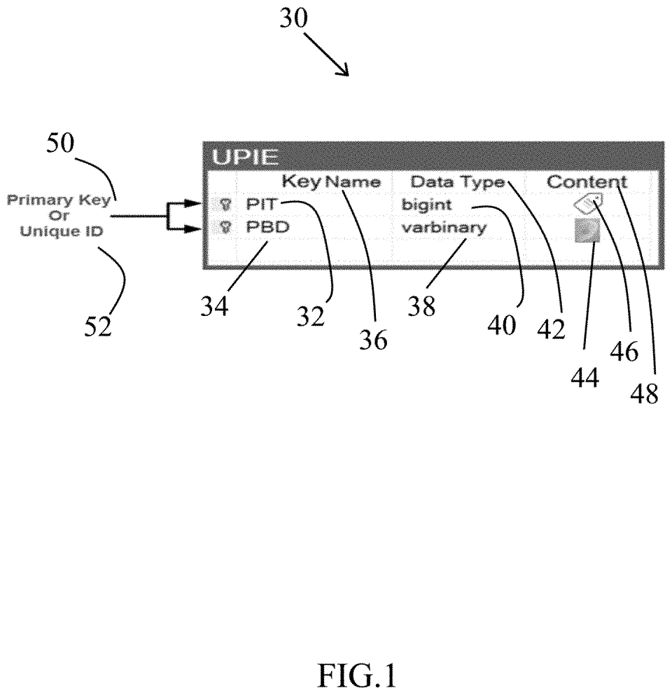

Referring now to FIG. 1, as the example outlined for healthcare use, illustrated is The Unique Patient Identification Entity (UPIE) 30 having a PIT 32, as well as patient's biometric data (PBD) 34, where 44 is ear biometric data that is used to uniquely identify each person. However, uniqueness is only guaranteed when the PIT 32 and PBD 34 are combined. This combination can be used in the nationwide Big Table as a Primary Key (PK) 50 or unique ID 52 which consists from two columns PIT 32 and PBD 34. Each of their column is Candidate Key that can uniquely identify a patient's record in a Big Table. The PIT 32 column can be in integer-based data type 42 with indexing that improves the speed of data retrieval operations to make select query run faster. Shown are data type 42, bigint 40, and varbinary 38. Key Name 36 is shown. Content 48 has PIT 32, Content 44, and biometric data Content 48.

Because each single column's data can be duplicated but the combination values of these columns cannot be duplicated, the searching by integer-based data in the first column of PIT 32 will reduce the amount of records for searching binary-based data in second column of PBD 34 data. The combination of PIT 32 and PBT 34 values called Unique Patient Identification Entity (UPIE) 30 can be used in the various types of relational and non-relational databases.

The Unique Patient Identification Entity (UPIE) 30 is both PIT 32 and PBT 34 together, forms pair as unique identity entity of person that uniquely identifies them among all other personal entities. Because value objects are immutable, we automatically fulfill the first requirement of Identity: immutability. Because both values of objects as a pair are unique they give us the second component of Identity: uniqueness. Thereby, UPIE 30 is immutable and globally unique.

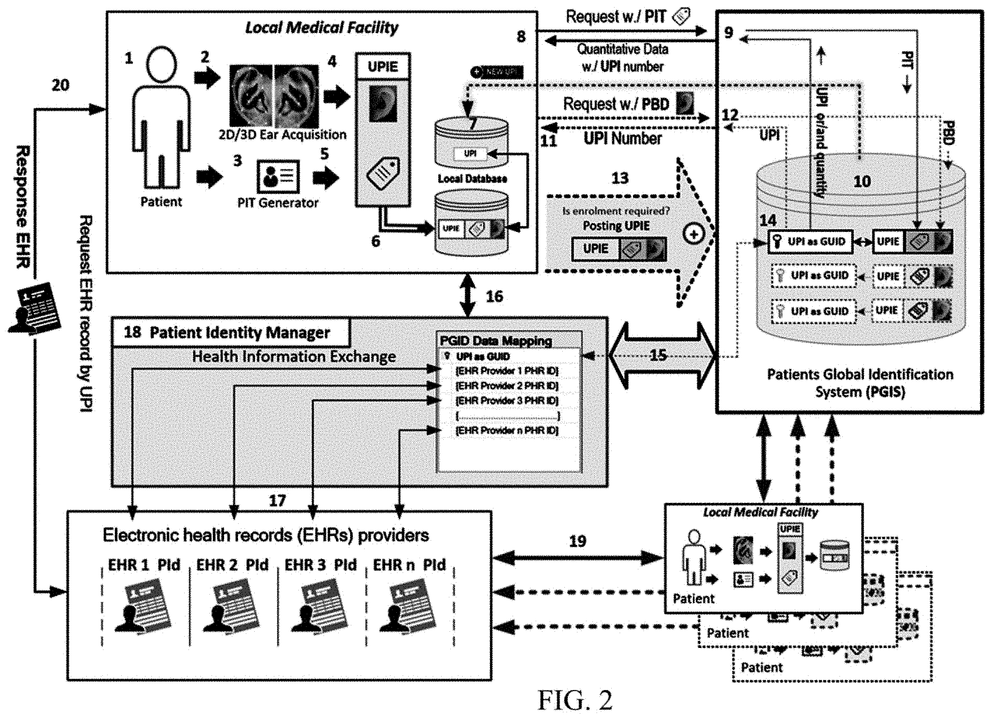

Referring now to FIG. 2 illustrated is Patients Global Unique Identification System (PGUIS). The uniqueness of this system architecture can be seen as a simple cost-effective way to managing patient identity across data source and the Unique Patient Identification Entity (UPIE) can be formed independently of the global system in any remote location, for example, in a medical facility. The images of a person's (patient's) ears (1) are captured with high-resolution camera and stored in the files. The ear region of each image is located and extracted by the recognition system (2). On the next phase the texture of the ear image is extracted (4) using linear time-invariant (LTI) filter responses and converts it to virtual extraction feature (acquired bio-metric data) for right ear and left ear, respectively. Extraction features of both right and left ears paired together to the patient binary biometric data file (PBD).

Meanwhile, patient's application data is processed by the program that calculates a PIT. When PIT and PBD objects are created they are combined in the unique person identification entity (UPIE). The UPIE is stored in the local database (7).

Patient identity management (PIM). Electronic health care data are constantly being generated and linked across multiple systems, including electronic health records (EHRs), patient registries, and claims databases. In general, every system assigns its own identifier to each patient whose data it maintains. This makes it difficult to track patients across multiple systems and identify duplicate patients when different systems are linked. Efforts to address this challenge are complicated by the need to protect personal privacy and security.

Several standards development organizations are involved in the development of PIM strategies and standards. Major organizations currently include:

Integrating the Healthcare Enterprise;

Health Level Seven International;

The Regenstrief Institute, Inc.

The Patient identity management (PIM) system. PGUIS can be used as the foundational platform for a centralized countrywide level electronic database of all patients known as Central Data Repository (CDR) (containing all relevant data including biometrics in uniquely designed formats, all-time updated) for global level identity management.

To obtain a Unique Patient Identification (UPI) number from the global database, a special lightweight search algorithm is used.

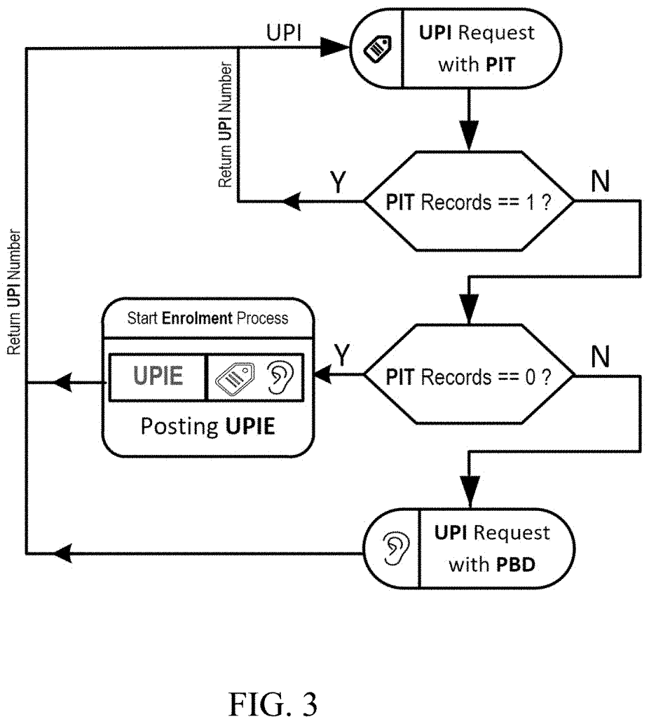

Referring now to FIG. 3 which represents illustrated a following logic diagram showing the flowchart of the lightweight protocol to obtain the UPI number from PGIS.

The local medical facility application sends PIT (8) to the global patient system (PGIS) as an integer number (9). Because the PIT number itself can be unique the PGIS response time to a request will be short. The PGIS fast response consists of small value of data (such as XML, JSON, or plain text) of two numbers: 1) Total number of records in the PGIS Bigtable for requested PIT number, and 2) the GUID (Globally Unique ID) number of PIT record in the PGIS Bigtable with the following variables: 1) if total number of records for requested PIT number equal 1, or 2) zero (null), or 3) if total number of records is not equal to 1. Depending on the value of the received total number of records in the PGIS Bigtable for requested PIT number, the following procedures are performed:

If total number of records in the PGIS Bigtable for requested PIT number equals to zero, the person enrolment process is performed: local medical facility sends UPIE entity (13) to the PGIS and retrieve response with UPI number (10). If total number of records in the PGIS Bigtable for requested PIT number equals to 1, the local medical facility receive response with UPI number (11). If total number of records in the PGIS Bigtable for requested PIT number more then 1, the local medical facility sends request with PBD (12) and receive response with UPI number (11).

When the number of UPI have been obtained, local facility application updates UPIE record of local database with (7) UPI and utilizes UPI to make a request to EHR companies and providers (16) of electronic health records (EHR) by UPI.

On this proposed approach, patient identity management system (PIMS) is part of the PDGUIS. The PIMS database contains records in which the unique patient identifier corresponds to the local identification number of patient health record on the provider's site.

While the present invention has been related in terms of the foregoing embodiments those skilled in the art will recognize that the invention is not limited to the embodiments described. The present invention may be practiced with modification and alteration within the spirit and scope of the appended claims. Thus, the description is to be regarded as illustrative instead of restrictive on the present invention.

The electronic health care providers (17), see FIG. 2, make additional check points for EHR using direct connection to the health information exchange system HIES (15). The health information exchange system (HIES) intended to share electronic patient health (ePHI) record across disparate health care applications. This platform provides inter-mobility to exchange patient data produced by health care applications (19) with other applications that consume and use the data, such as EHRs.

What is claimed is:

1. A personal tag comprising:
a specific integer number formed by converting a person's full name, birthday and eye color by special following algorithm;
a personal full name string is a set of names by which an individual is known and is a word-group, the word group relates to that one individual, the full name is first or given, middle, and last or family or surname;
remove all non-alphanumeric characters from the full name string;
convert to uppercase the full name string value;
a first character's coefficient number is calculated as the sum of the ASCII values of first letters of each word in the full name string in English Speaking Countries, or calculated based on cultural alternative characters table for other countries;
from the full name string remove all spaces;
a first part of a person's TAG number is a sum of ASCII value of each character in a full name string;
a second part of the person's TAG number has a string join of values from a day number plus a month number and a year of the birthday.
a third part of the person's TAG number is formed from a length value of the full name string plus first character's coefficient number;
a forth part of the person's TAG number is an eye color code based on eye color chart: BLK Black=1, BLU Blue=2, BRO Brown=3, GRY Gray=4, GRN Green=5, HAZ Hazel=6, MAR Maroon=7, PNK Pink=8, DIC Dichromatic=9, SPC Spectrum=10.

2. Ear biometric data comprising:
analysis of a curve of the ear's helix to be a most reliable anatomical structure antihelix, tragus, antitragus, inter-tragal incisura, and an ear lobule pair both right and left ears together.

3. A unique identification of a person, or patient comprising:
person's identity tag; and
person's biometric data.

4. A Unique Person Identification number from a global database, a special lightweight search algorithm comprising:
a local service facility application where person's identity service is performed sends PIT to a global database storage system as an integer number having total number of records in a PGIS Bigtable for requested PIT number; and
the GUID (Globally Unique ID) number of PIT record in the PGIS Bigtable, if total number of records for requested PIT number equals 1, or zero (null), or if total number of records is not equal to 1;

5. A unification system that allows countrywide, or global management of personal data between many other personal data management systems in healthcare having a platform comprising of the following steps:
a. Provision of a UPI number;
b. Personal identity source (PIS) (e.g. local service facility application) obtains the UPI and updates UPIE record of local database with unique patient identifier UPI from PGUIS;
c. PIS utilizes UPI and makes a request to personal identifier cross-reference manager (e.g. EHR companies and providers in health care) of electronic personal records (EPR) by UPI;
d. The electronic health care providers make additional check points in the EHR system using direct connection to the health information exchange system. The health information exchange system (HIES) intends to share electronic patient health (ePHI) record across disparate health care applications and provides inter-operability to exchange patient data produced by PIS with other PIS(s) which consume and use data (e.g. such as EHRs).

6. A system and method of integrated person unique identity management with two independent identification data objects comprising:
PIT transformed from personal individual data, and
biometric data.

7. The system and method of claim 6 wherein a person's tag is a transformation of the personal individual data, the personal individual data is transformed into a specific integer number, the specific integer number is formed by transforming the personal individual data into the specific integer number using a special algorithm.

8. The system and method of claim 7 wherein the personal individual data is a person's full name, birthday and eye color, the special algorithm transforms the personal individual data into the specific integer number, the specific integer number has a first part, a second part, a third part, and a fourth part.

9. The system and method of claim 8 wherein the first part of the specific integer number is a sum of ASCII, or similar, values of each character in a full name string.

10. The system and method of claim 8 wherein the second part of the specific integer number is a string join of values from a day number plus a month number and a year of a patent's birthday arranged with a four-digit year first then a day then a month from left to right.

11. The system and method of claim 8 wherein the third part of the specific integer number is formed from a length value of a patent's full name string plus a first character's coefficient number.

12. The system and method of claim 8 wherein the forth part of the person's tag number is specific integer number is eye color code based on an eye color chart.

13. The system and method of claim 12 wherein the eye chart is blk black=1, blu blue=2, bro brown=3, gry gray=4, grn green=5, haz hazel=6, mar maroon=7, pnk pink=8, dic dichromatic=9 and spc spectrum=10

14. The system and method of claim 6 wherein the biometric data is ear biometric data the ear biometric data is analysis of a curve of a ear's helix, antihelix, tragus, antitragus, inter-tragal incisura, and an ear lobule paired both, right and left ears together.
